(19) United States
(12) Patent Application Publication
(10) Pub. No.: US 2013/0331936 A1
(43) Pub. Date: Dec. 12, 2013

(54) INTRAOCULAR IMPLANT

(71) Applicant: Francesco FERRARI, Strasbourg (FR)

(72) Inventor: Francesco FERRARI, Strasbourg (FR)

(21) Appl. No.: 13/552,912

(22) Filed: Jul. 19, 2012

(30) Foreign Application Priority Data
Jun. 6, 2012 (FR) 12 55292

(51) Int. Cl. A61F 2/16

(52) U.S. Cl. 623/6.27; 623/6.39

(57) ABSTRACT

An embodiment of the intraocular implant includes two portions, a single-focus or multi-focus optical central portion, and a haptic peripheral portion. The haptic peripheral portion is formed of two extensions extending diametrically with respect to the optical central portion, aimed at immobilizing the implant in the capsular bag by applying their end portions against the edges of the capsular bag, on the inner side thereof. The extensions forming the haptic portion each extend along a plane that is inclined with respect to the main plane of symmetry of the optic portion, and according to inclinations in opposite directions.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates to an intraocular implant for use in the field of the cataract surgery.

It is recalled that the cataract surgery consists of replacing the opaque crystalline of the eye with an artificial crystalline, i.e. an intraocular implant that is placed in the capsular bag.

2. Description of Related Art Including Information Disclosed Under 37 CFR 1.97 and 37 CFR 1.98.

Generally, an intraocular implant comprises two portions, a central portion, referred to as optical portion, which focuses the light onto the retina and is single-focus or multi-focus, and a peripheral portion, referred to as haptic portion, formed of diametrically opposed extensions, aimed at immobilizing the implant in the capsular bag by applying its end portions against the edges of said capsular bag, internally to same.

There are essentially two types of intraocular implants, such as those represented in FIGS. 1, 2, 3 and 4, namely the shuttle implants 1 visible in FIGS. 1 and 2, and the implants with lugs 2, visible in FIGS. 3 and 4.

A shuttle implant 1, represented in plan view in FIG. 2 and in elevation view in FIG. 1, consists of an optical portion 10 in the form of a lens arranged at the center of a rectangular plate 11, the end portions 12 of which forming diametrically opposed extensions with respect to the optical portion 10 and aimed at immobilizing the implant 1 in the capsular bag.

An implant with lugs 2, represented in plan view in FIG. 4 and in elevational view in FIG. 3, essentially comprises an optical portion 20 in the form of a lens, from which extend, symmetrically with respect to the center of the optical portion 20, two flexible lugs 21 forming open loops, and constituting the haptic portion aimed at immobilizing the implant 1 in the capsular bag.

In one eye, the cornea, the pupil and the crystalline have different axes of symmetry, which permit to define a median axis, which is referred to as “Best Fit Axis” (BFA), and there exists an angular distance between this axis and the visual axis, which is generally convergent, the angular distance between these two axes being referred to as kappa angle.

Though in the field of refractive surgery the kappa angle is taken into consideration in the restoration of the vision, this is not the case in cataract surgery, so that the vision after an operation of cataract surgery is not restored optimally.

### SUMMARY OF THE INVENTION

The object of the present invention is to cope with the aforementioned drawback by providing an intraocular implant that can take the angle kappa into consideration.

The intraocular implant according to the invention comprises two portions, a single-focus or multi-focus optical central portion, and a haptic peripheral portion, and formed of two extensions extending diametrically with respect to said optical portion, aimed at immobilizing the implant in the capsular bag by applying their end portions against the edges of said capsular bag, on the inner side thereof, and it is essentially characterized in that said extensions constituting said haptic portion, each extending in a plane, which is inclined with respect to the main plane of symmetry of said optical portion, and according to inclinations in opposite directions.

It will be understood that the inclination of the extensions constituting the haptic portion with respect to the optical portion permits to place the latter as perpendicular as possible to the visual axis, so as to angularly correct orientation of the optical portion with an angle corresponding to kappa angle.

According to an additional feature of the intraocular implant of the invention, the extensions have unequal lengths.

The difference in length of the extensions permits to place the optical portion off center in the capsular bag, in order to center it on said visual axis.

Thus, the combination of the inclination of the extensions and their difference in length permits to optimally correct the positioning of the optical portion.

According to another additional feature of the intraocular implant of the invention, the longest extension is aimed at being positioned on the temporal side.

According to another additional feature of the intraocular implant of the invention, it is made of a single piece.

According to another additional feature of the intraocular implant of the invention, it is made of a hydrophilic material.

According to another additional feature of the intraocular implant of the invention, it is made of a hydrophobic material.

According to a particular embodiment of the intraocular implant according to the invention, the extensions forming the haptic portion consist of lugs forming open loops.

According to another particular embodiment of the intraocular implant of the invention, it is of the shuttle type and the extensions forming the haptic portion consist of flat walls.

The advantages and features of the intraocular implant of the invention will become clear from the following description, which refers to the attached drawing, which represents a non-restrictive embodiment of same.

### BRIEF DESCRIPTION OF THE DRAWINGS

FIG. 1 shows a schematic elevational view of a shuttle-type intraocular implant.

FIG. 2 shows a schematic plan view of the same shuttle-type intraocular implant.

FIG. 3 shows a schematic elevational view of an intraocular implant with lugs.

FIG. 4 shows a schematic plan view of the same intraocular implant with lugs.

FIG. 5 shows a schematic plan view of an intraocular implant with lugs according to the invention.

FIG. 6 shows a schematic elevational view of the same intraocular implant with lugs according to the invention.

FIG. 7 shows a schematic plan view of a shuttle-type intraocular implant according to the invention.

FIG. 8 shows a schematic elevational view of the same shuttle-type intraocular implant according to the invention.

FIG. 9 shows a schematic top and median cross-sectional view of part of a state-of-the-art implant implanted in a right eye.

FIG. 10 shows a schematic top and median cross-sectional view part of an implant according to the invention implanted in a right eye.

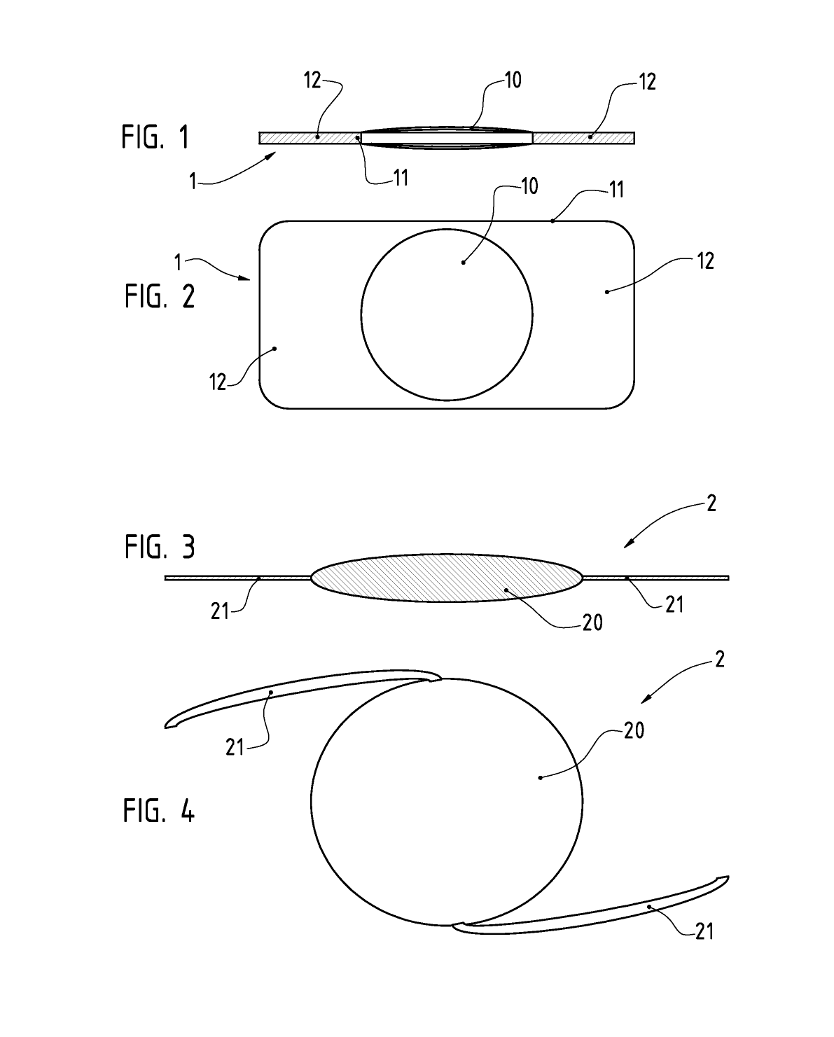

### DETAILED DESCRIPTION OF THE DRAWINGS

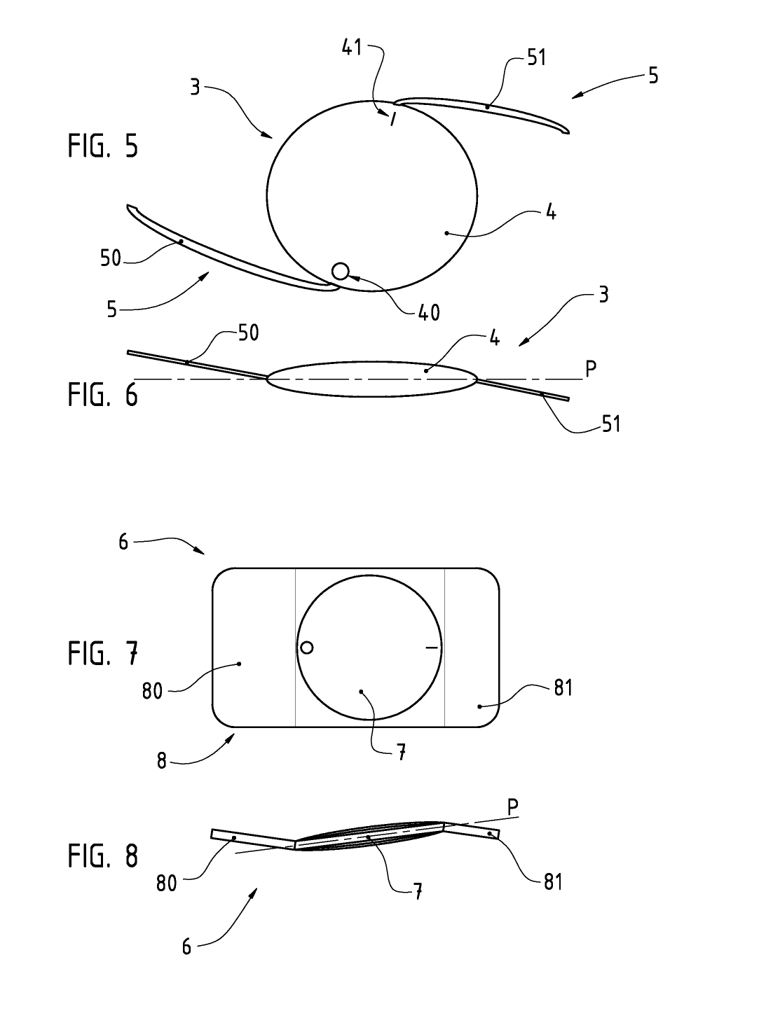

When referring to FIGS. 5 and 6, one can see an intraocular implant 3 according to the invention. In a way known per se, this ocular implant 3 includes an optical portion 4 in the form of a lens, and a haptic portion 5 formed of two lugs 50 and 51.

According to the invention the lugs 50 and 51 are inclined with respect to the main plane of symmetry P of the optical portion 4, one 50 towards the rear of the intraocular implant 3, and the other 51 towards the front.

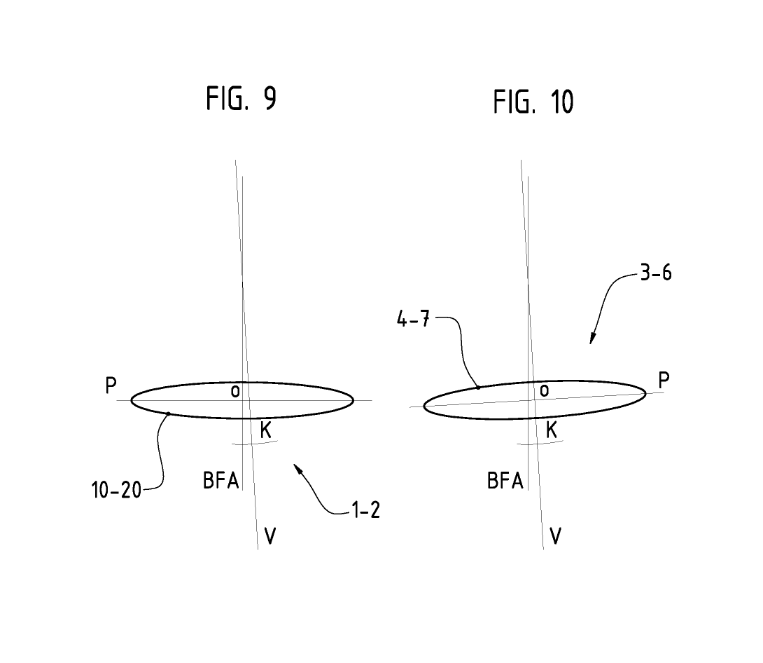

Thus, the optical portion 4 is inclined in the intraocular 3, whereby this inclination can be varying according to the inclinations of the lugs 50 and 51 with respect to the plane P. The orientation of the optical portion 4 permits to place it perpendicular to the visual axis, as will be explained hereinafter, by means of FIGS. 9 and 10.

One can also see in these FIGS. 5 and 6 that the lugs 50 and 51 are of different lengths. This difference in length is aimed at placing the optical portion 4 off center in the capsular bag, so as to center it on the visual axis.

It should be noted that the optical portion 4 has two different marks 40 and 41, each facing one of the lugs 50 and 51, so as to facilitate the positioning of the implant in the capsular bag 3, knowing that the difference in length of the lugs 50 and 51 is in the range of one millimeter, and that the inclination can be in the range of one degree.

Generally, the visual angle is convergent, so that to re-center the optical portion 4 on this visual axis, the lug 50, aimed at being positioned in the capsular bag on the temporal side, is longer than the lug 51.

Each of the characteristics of the lugs 50 and 51, namely on the one hand their inclination with respect to the plane P, and on the other hand their difference in length, is aimed at bringing a correction depending on the value of the kappa angle, knowing that the optimal correction is achieved by combining the two characteristics.

It should be noted that there are special cases, in which one observes a shift of the pupil. Therefore, it is foreseen to use an intraocular implant 3, the lengths of the lugs 50 and 51 of which are adapted for also correcting this discrepancy, however knowing that, because such a shift of the pupil is in most cases temporal, the total correction cannot be optimal.

When referring to FIGS. 7 and 8, one can see an intraocular lens 6 of the invention, in a shuttle-type version, and which thus comprises an optical portion 7 in the form of a lens arranged at the center of a rectangular plate forming the haptic portion 8, and the end portions 80 and 81 of which extend diametrically on both sides of the optical portion 7, are aimed at positioning the intraocular implant in the capsular bag. According to the invention, the end portions 80 and 81 form an angle with the main plane of symmetry of the optical portion 7, and are of different lengths.

When referring now to FIGS. 9 and 10, one can see the effects of the correction made by an intraocular implant 3 or 6.

In FIG. 9, there is shown the optical portion 10 or 20 of a conventional intraocular implant 1 or 2, respectively, and one can see that this optical portion 10 or 20 is centered on the BFA axis that passes through its center ◯, while the visual axis V separates angularly from same by an angle K.

In FIG. 10, one can see the optical portion 4 or 7 of an intraocular implant 3 or 6, respectively, and one can observe that the double correction made by the haptic portion, not shown, i.e. on the one hand the inclination of the optical portion 4 or 7, and on the other hand its lateral offset through the differences in length of the lugs or the end portions, permits that the visual axis V passes through the center ◯ of the optical portion 4 or 7, and perpendicularly to the plane P.

It should be noted that it is possible to imagine intraocular lenses according to the invention, the haptic portion of which is perfectly symmetrical in dimensional, i.e. the optical portion of which is corrected only angularly, in this case the visual axis passes perpendicularly through the plane of symmetry P of the optical portion and passes in the vicinity of the center ◯ of the latter.

Furthermore, the present invention is not limited to the only embodiments described above, knowing that the lugs of the intraocular implants can have many other configurations, as well as the extensions of shuttle-type intraocular implant.

1. Intraocular implant comprising:
a single-focus or multi-focus optical central portion; and
a haptic peripheral portion being comprised of two extensions, extending diametrically with respect to said optical central portion, being aimed at immobilizing portions in a capsular bag by applying end portions of the haptic portion against edges of said capsular bag, on the inner side thereof,
wherein said two extensions forming said haptic portion each extend along a plane inclined with respect to a main plane of symmetry of said optic central portion, and in accordance to inclinations in opposite directions.

2. Intraocular implant according to claim 1, wherein the extensions have unequal lengths.

3. Intraocular implant according to claim 2, wherein a longer extension is aimed at being positioned on a temporal side.

4. Intraocular implant according to claim 1, wherein the portions are made integral in one single piece.

5. Intraocular implant according to claim 1, wherein the portions are comprised of a hydrophilic material.

6. Intraocular implant according to claim 1, wherein the portions are comprised of a hydrophobic material.

7. Intraocular implant according to claim 1, wherein the extensions forming the haptic portion are comprised of lugs forming open loops.

8. Intraocular implant according to a claim 1, wherein the extensions forming the haptic portion are comprised of flat walls, being of a shuttle-type of implant.
